(19) United States
(12) Patent Application Publication
(10) Pub. No.: US 2025/0339147 A1
(43) Pub. Date: Nov. 6, 2025

(54) SURGICAL STAPLER WITH UNIVERSAL TIP RELOAD

(71) Applicant: Covidien LP, Mansfield, MA (US)

(72) Inventors: Pawel Abramek, Berlin, CT (US); Kenneth M. Cappola, Monroe, CT (US); Matthew J. Chowaniec, Madison, CT (US); Zalika Walcott, Hamden, CT (US)

(73) Assignee: Covidien LP, Mansfield, MA (US)

(21) Appl. No.: 19/270,708

(22) Filed: Jul. 16, 2025

Related U.S. Application Data
(63) Continuation of application No. 16/713,345, filed on Dec. 13, 2019, now Pat. No. 12,383,260.

(51) Int. Cl. A61B 17/068; A61B 17/072; A61B 17/32

(52) U.S. Cl. CPC A61B 17/0684; A61B 2017/07257; A61B 2017/07285; A61B 2017/320044

(57) ABSTRACT

A surgical stapler includes a tool assembly that has a cartridge assembly, an anvil assembly, and a dissector tip. The dissector tip is moveable to define at least two angled configurations of the dissector tip, relative to a longitudinal axis through the tool assembly, for use during a surgical procedure.

### CROSS-REFERENCE TO RELATED APPLICATIONS

This application is a continuation of U.S. patent application Ser. No. 16/713,345, filed Dec. 13, 2019, the entire disclosure of which is incorporated by reference herein.

### FIELD

The disclosure is directed to surgical stapling devices and, more particularly, to endoscopic surgical stapling devices.

### BACKGROUND

Surgical stapling devices for performing surgical procedures endoscopically are well known. Such devices are available in a variety of different configurations, e.g., linear, curved, circular, etc., and are suitable for use in a variety of different procedures. Linear surgical stapling devices include a tool assembly having an anvil and a staple cartridge that are pivotably coupled to each other at their proximal ends between open and clamped positions. In order to better navigate the tool assembly to a surgical site endoscopically, the tool assembly may include a dissector tip that extends from a distal end of the tool assembly. Typically, the dissector tip is supported on the tool assembly and is configured to separate target tissue from body tissue to facilitate placement of the tool assembly about the target tissue. During some surgical procedures, it is desirable to have an angled dissector tip whereas in other surgical procedures it is desirable to have a linear dissector tip.

A continuing need exists in the art for a surgical stapling device that is better suited to access to a variety of surgical sites.

### SUMMARY

One aspect of this disclosure is directed a surgical stapler including an elongate body and a tool assembly. The elongate body has a proximal portion and a distal portion. The tool assembly defines a longitudinal axis and includes a cartridge assembly, an anvil assembly, and a dissector tip. The anvil assembly has a proximal portion and a distal portion and is coupled to the cartridge assembly such that the tool assembly is moveable from an open position to a clamped position. The dissector tip includes a body having a proximal end and a distal end. The body has a thickness that decreases towards the distal end. The body is movably coupled to the anvil assembly for movement between a first configuration substantially aligned with the longitudinal axis and a second configuration defining an acute angle with the longitudinal axis.

In aspects of the disclosure, the dissector tip includes a tip beam having a proximal portion and a deformable distal beam portion, wherein the deformable distal beam portion is bendable between the first and second configurations.

In some aspects of the disclosure, the distal portion of the anvil assembly includes a bracket and the proximal portion of the tip beam is secured to the bracket.

In certain aspects of the disclosure, the proximal portion of the tip beam is secured to the bracket of the anvil assembly by welding or other means (mechanical snap, adhesive, pin, etc).

In aspects of the disclosure, the dissector tip includes a tip beam cover that is supported on the deformable distal beam portion and has an atraumatic configuration.

In some aspects of the disclosure, the tip beam cover is formed of a biocompatible material selected from the group consisting of polymeric materials, metals, ceramics, and elastomeric materials.

In certain aspects of the disclosure, the dissector tip includes a body having a proximal portion and a distal portion, wherein the proximal portion includes a hinge portion that is pivotably coupled to the distal portion of the anvil assembly.

In aspects of the disclosure, the distal portion of the anvil assembly includes a clevis and the hinge portion is coupled to the clevis by a clevis pin.

In some aspects of the disclosure, the tool assembly includes retaining structure configured to retain the dissector tip in one of the first and second configurations.

In certain aspects of the disclosure, the retaining structure includes a protrusion formed on one of the distal portion of the anvil assembly or the proximal portion of the dissector tip and a plurality of recesses formed on the other of the distal portion of the anvil assembly or the proximal portion of the dissector tip.

In one aspect of the disclosure, the dissector tip has a width that decreases towards the distal end of the dissector tip.

Another aspect of the disclosure is directed to a tool assembly including a cartridge assembly, an anvil assembly, and a dissector tip. The anvil assembly includes a proximal portion and a distal portion and is coupled to the cartridge assembly such that the tool assembly is moveable from an open position to a clamped position. The tool assembly defines a longitudinal axis. The dissector tip includes a body having a proximal end and a distal end and has a thickness that decreases towards the distal end. The body is movably coupled to the anvil assembly for movement between a first configuration substantially aligned with the longitudinal axis and a second configuration defining an acute angle with the longitudinal axis.

Another aspect of the disclosure is directed to a tool assembly defining a longitudinal axis and including a cartridge assembly, an anvil assembly, and a dissector tip. The anvil assembly includes a base portion and an anvil portion. The base portion is coupled to the cartridge assembly such that the tool assembly is moveable from an open position to a clamped position. The anvil portion is secured to the base portion and includes a plurality of staple deforming pockets. The anvil portion includes a distal portion supporting a bracket. The dissector tip includes a body having a tapered distal surface and a thickness that decreases in the distal direction. The body is movably coupled to the anvil assembly for movement between a first configuration substantially aligned with the longitudinal axis and a second configuration defining an acute angle with the longitudinal axis. The body of the dissector tip includes a tip beam having a proximal portion and a deformable distal beam portion. The proximal portion of the dissector tip is secured to the bracket of the anvil assembly and the deformable distal beam portion is bendable between the first and second configurations.

Other features of the disclosure will be appreciated from the following description.

### BRIEF DESCRIPTION OF THE DRAWINGS

Various aspects of the disclosed surgical stapling device are described herein below with reference to the drawings, wherein:

FIG. 1 is a side perspective view of exemplary aspects of the disclosed surgical stapling device including a reload assembly with a universal tip;

FIG. 2 is an enlarged view of the indicated area of detail shown in FIG. 1;

FIG. 3 is a perspective bottom view of an anvil assembly of the reload assembly of the surgical stapling device shown in FIG. 1;

FIG. 4 is an exploded, perspective view of the anvil assembly shown in FIG. 3;

FIG. 5 is an enlarged cutaway, perspective view of a distal portion of an anvil body and tip beam of the anvil assembly shown in FIG. 4;

FIG. 6 is a side perspective view of a tool assembly of the reload assembly shown in FIG. 1 in a clamped position;

FIG. 7 is a cross-sectional view taken along section line 7-7 of FIG. 6 with the anvil tip in a first position;

FIG. 8 is a cross-sectional view taken along section line 7-7 of FIG. 6 with the anvil tip in a second position;

FIG. 9 is a cross-sectional view taken along section line 7-7 of FIG. 6 with the anvil tip in a third position;

FIG. 10 is a side view of other exemplary aspects of a tool assembly of the reload assembly of the stapling device shown in FIG. 1 with the tool assembly in the clamped position;

FIG. 11 is an enlarged side perspective view of a distal end of the tool assembly shown in FIG. 10 with the tool assembly in the clamped position;

FIG. 12 is a side perspective, exploded view of the anvil assembly of the tool assembly shown in FIG. 10;

FIG. 13 is an enlarged view of the distal end of the anvil base of the anvil assembly shown in FIG. 10;

FIG. 14 is a perspective view from the proximal end of the dissector tip of the anvil assembly shown in FIG. 11;

FIG. 15 is a cross-sectional view taken along section line 15-15 of FIG. 11 with the dissector tip in a first position; and

FIG. 16 is a cross-sectional view taken along section line 15-15 of FIG. 11 with the dissector tip in a second position.

### DETAILED DESCRIPTION

The disclosed surgical stapling device will now be described in detail with reference to the drawings in which like reference numerals designate identical or corresponding elements in each of the several views. However, it is to be understood that the aspects of the disclosure described herein are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician, while the term “distal” is used generally to refer to that portion of the device that is farther from the clinician. In addition, the term “endoscopic” is used generally to refer to endoscopic, laparoscopic, arthroscopic, and/or any other procedure conducted through a small diameter incision or cannula. Further, the term “clinician” is used generally to refer to medical personnel including doctors, nurses, and support personnel. As used herein, the terms “parallel” and “aligned” are understood to include relative configurations that are substantially parallel, and substantially aligned, .i.e., up to about + or −10 degrees from true parallel or true alignment.

The disclosed surgical stapling device includes a tool assembly that supports a dissector tip that is adjustable to allow a clinician to position the dissector tip in a configuration that facilitates easy access to a surgical site. Some aspects of the disclosure are directed to a dissector tip that is bendable between a first orientation in which an axis defined by dissector tip is parallel to a longitudinal axis of the tool assembly and a second position in which the axis defined by the dissector tip defines an acute angle with the longitudinal axis of the tool assembly. Other aspects of the disclosure are directed to a dissector tip that is pivotable between a first orientation in which the axis defined by the dissector tip is parallel to a longitudinal axis of the tool assembly and a second position in which the axis defined by the dissector tip defines an acute angle with the longitudinal axis of the tool assembly.

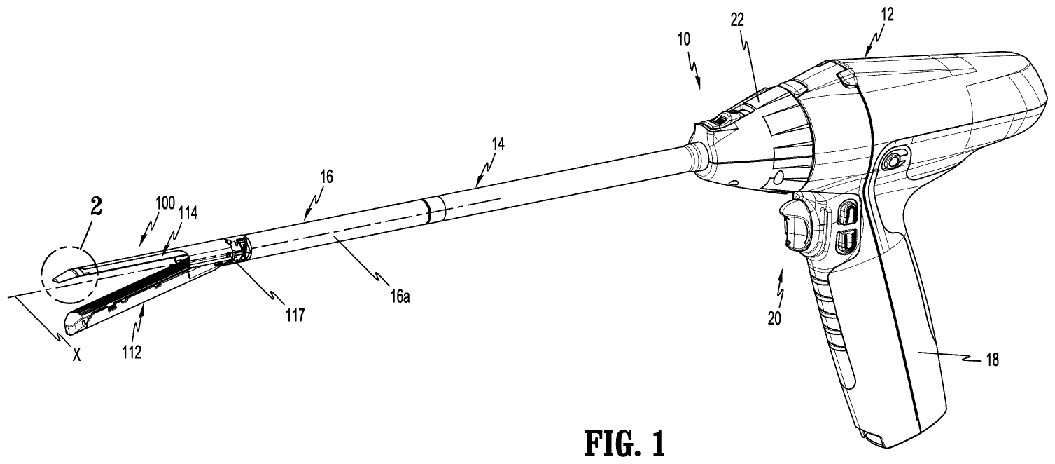

FIG. 1 illustrates the disclosed surgical stapling device 10 including a handle assembly 12, an elongate body 14, and a tool assembly 100. Although not described in detail herein, the tool assembly 100 can form part of a reload assembly 16 that is releasably coupled to the elongate body 14 and includes a proximal body portion 16a and the tool assembly 100. Alternately, the tool assembly 100 can be fixedly secured to a distal portion of the elongate body 14. The handle assembly 12 includes a hand grip 18, a plurality of actuator buttons 20, and a rotation knob 22. The rotation knob 22 facilitates rotation of the elongate body 14 and the tool assembly 100 in relation to the handle assembly 12. The actuator buttons 20 control operation of the various functions of the stapling device 10 including approximation, firing and cutting. Although the stapling device 10 is illustrated as an electrically powered stapling device, it is envisioned that the disclosed tool assembly 100 would also be suitable for use with a manually powered surgical stapling device. U.S. Pat. No. 9,055,943 (the '943 Patent) discloses a surgical stapling device including a powered handle assembly and U.S. Pat. No. 6,241,139 (the '139 Patent) discloses a manually actuated handle assembly. The tool assembly 100 includes a cartridge assembly 112, an anvil assembly 114, and a dissector tip 120. For a more detailed description of the cartridge assembly 112 of the tool assembly 100, see, e.g., the '943 and '139 Patents.

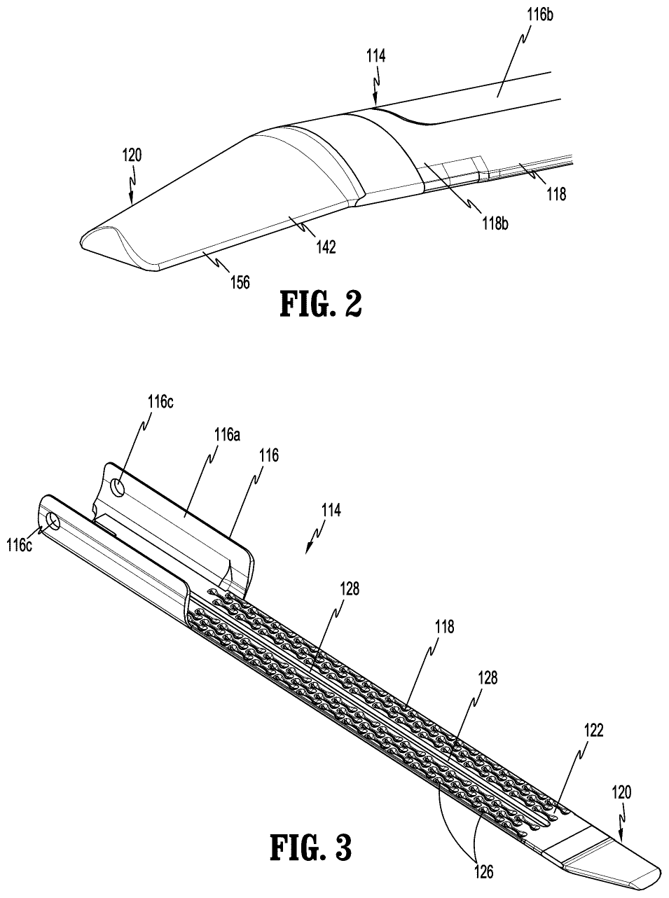

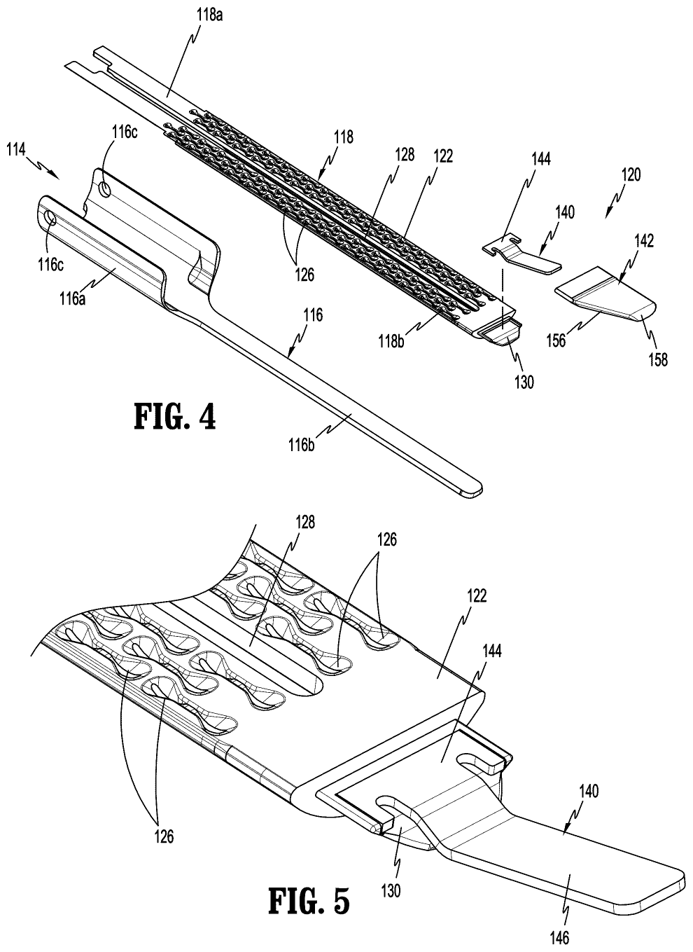

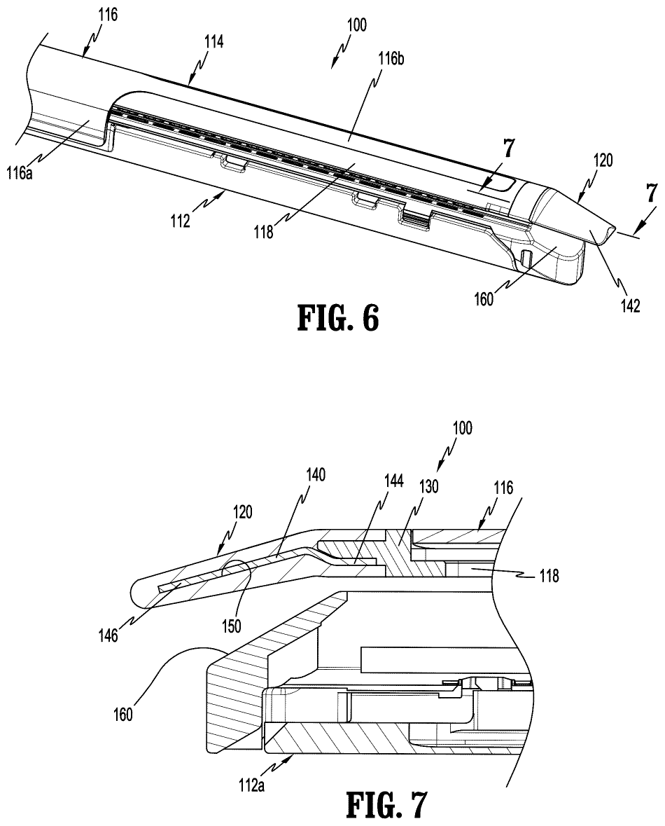

FIGS. 2-7 illustrate the anvil assembly 114 of the tool assembly 100 which defines a longitudinal axis “X” and includes a base portion 116 and an anvil portion 118. The base portion 116 includes a proximal mounting portion 116a and a longitudinal rib 116b that extends distally from the proximal mounting portion 116a. The proximal mounting portion 116a includes spaced through bores 116c that receive pivot members 117 (FIG. 1) that pivotably couple the cartridge assembly 112 to the anvil assembly 114. The anvil portion 118 is secured to the base portion 116 by, e.g., welding, and includes an upper surface 122, a proximal portion 118a, and a distal portion 118b. The upper surface 122 of the anvil portion 118 defines a plurality of rows of staple deforming pockets 126 that are positioned on opposite sides of a knife slot 128. The distal portion 118b of the anvil portion 118 includes a support plate or bracket 130.

The dissector tip 120 includes a tip beam 140 and a tip beam cover 142. The tip beam 140 includes a body having a proximal portion 144 and a deformable distal beam portion 146. The tip beam 140 is secured to the support bracket 130 on the distal portion 118b of the anvil portion 118 and is formed from a substantially rigid material that can be bent and shaped to a desired configuration such as a metal. The tip beam cover 142 has an atraumatic configuration and is formed of any suitable biocompatible material including polymeric materials, metals, ceramics, elastomeric materials, etc. In certain aspects of the disclosure, the tip beam cover 142 has rounded edges 156 and a tapered distal tip 158 (FIG. 4) that decreases in width in the distal direction. The tip beam cover 142 also has a thickness that decreases in the distal direction such that the cover 142 is thinnest at its distal end. The reduced thickness allows for the dissector tip 120 to more easily navigate through and dissect tissue. Other tip beam cover configurations are envisioned.

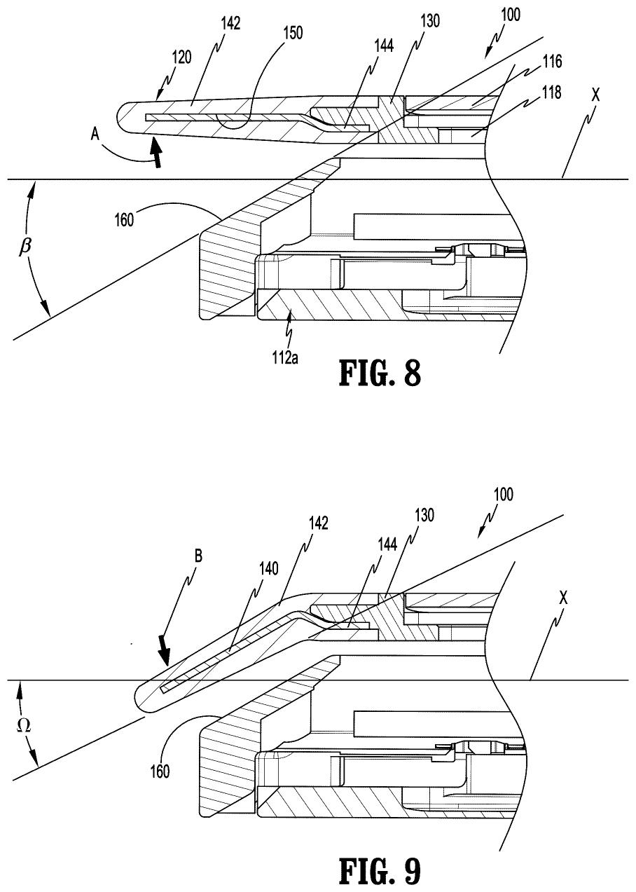

FIGS. 8 and 9 illustrate the distal portion of the tool assembly 100 as the dissector tip 120 is moved between a first configuration (FIG. 8) in which the dissector tip 120 extends in a direction substantially parallel to the longitudinal axis “X” of the tool assembly 100 and a second configuration (FIG. 9) in which the dissector tip 120 of the tool assembly 100 extends in a direction to define an acute angle Ω with the longitudinal axis “X” of the tool assembly 100. In certain aspects of the disclosure, a distal portion 112a of the cartridge assembly 112 defines a tissue guide surface 160 that defines an angle B with the longitudinal axis “X” of the tool assembly 100. In order to move the dissector tip 120 between the first and second configurations, the tip beam 140 and the tip beam cover 142 are manually grasped by a clinician and bent or deformed in the direction of arrows “A” or “B” in FIGS. 8 and 9, respectively, to a desired configuration best suited to perform a certain surgical procedure e.g., in which angle Ω is from about zero to about 45 degrees. The tip beam 140 is formed of a material that will retain its deformed configuration as the dissector tip 120 is used to separate target tissue from body tissue as the tool assembly 100 is advanced endoscopically to a surgical site.

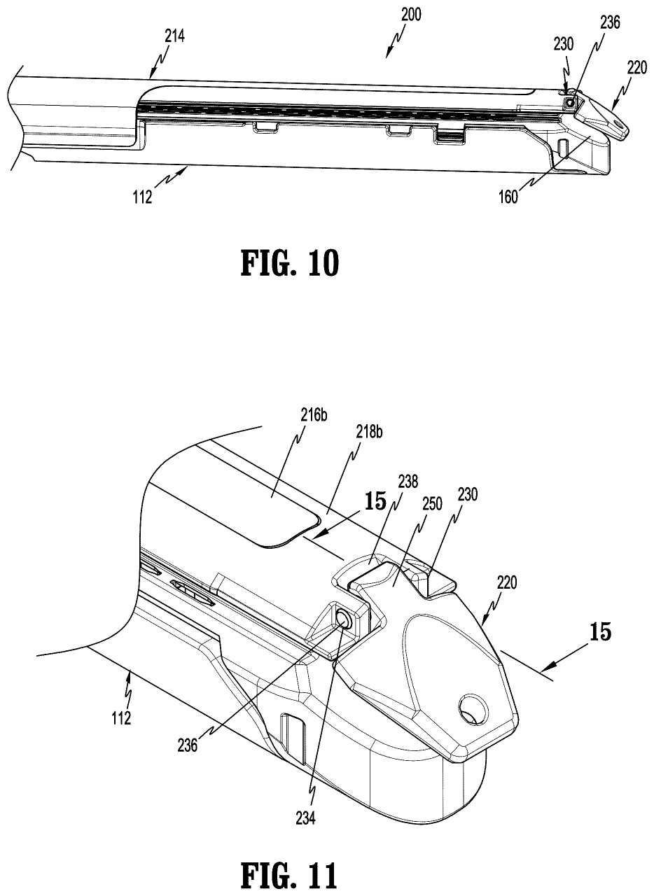

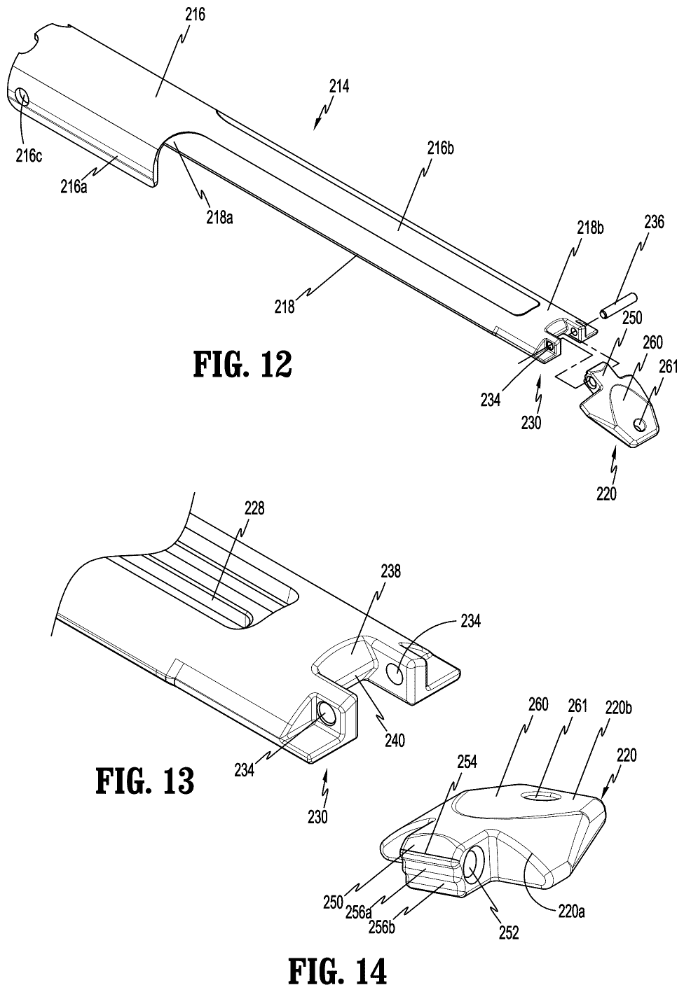

FIGS. 10-14 illustrate other exemplary aspects of a tool assembly 200 according to the disclosure. The tool assembly 200 includes an anvil assembly shown generally as anvil assembly 214. The anvil assembly 214 is similar to the anvil assembly 114 but includes modifications to the dissector tip 120. Only those modifications to the dissector tip shown generally as dissector tip 220 will be described in detail herein.

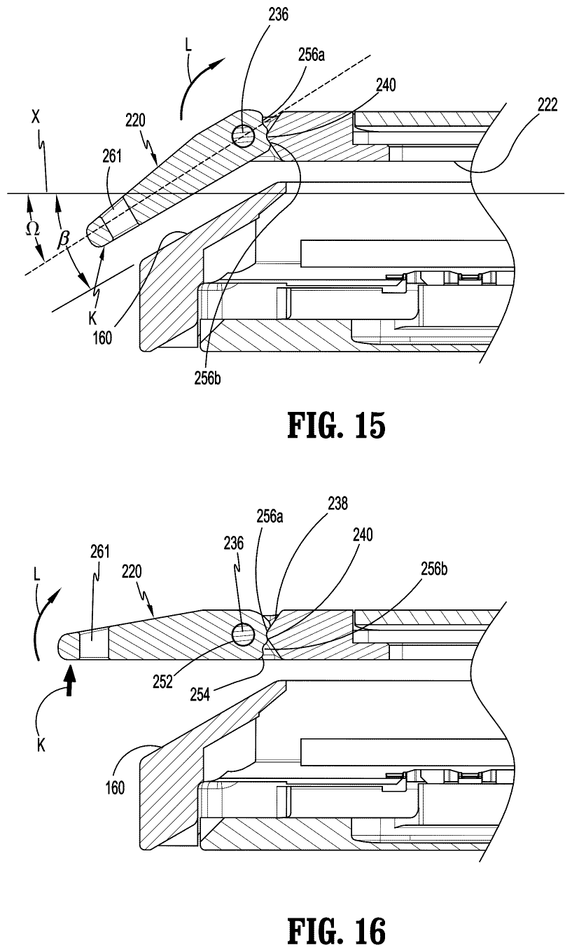

The anvil assembly 214 includes a base portion 216, an anvil portion 218, and the dissector tip 220. The base portion 216 includes a proximal mounting portion 216a and a longitudinal rib 216b that extends distally from the proximal mounting portion 216a towards a distal end of the anvil portion 218. The proximal mounting portion 216a includes spaced through bores 216c that receive pivot members 117 (FIG. 1) that pivotably couple the cartridge assembly 112 (FIG. 1) to the anvil assembly 214. The anvil portion 218 is secured to the base portion 216 by, e.g., welding, and includes an upper surface 222 (FIG. 15), a proximal portion 218a, and a distal portion 218b. The upper surface 222 of the anvil portion 118 defines a plurality of rows of staple deforming pockets (not shown) that are positioned on opposite sides of a knife slot 228 (FIG. 13). The distal portion 218b of the anvil portion 118 includes a clevis 230 that defines spaced openings 234 that receive a clevis pin 236. The distal portion 218b of the anvil portion 218 includes a distal face 238 that has a protrusion 240.

The dissector tip 220 has a body including a proximal portion 220a and a distal portion 220b. The proximal portion 220a includes a hinge portion 250 that defines a through bore 252 that receives the clevis pin 236 to pivotably secure the dissector tip 220 to the distal end portion 218b of the anvil portion 218. The dissector tip 220 has a proximal face 254 that defines spaced recesses 256a and 256b that are positioned to receive the protrusion 240 on the distal face 238 of the anvil portion 218 (FIG. 15). The dissector tip 220 has a tapered distal face 260 in the distal direction (FIG. 15) such that the distal end of the dissector tip 220 has a height that decreases in the distal direction. In addition, the width of the dissector tip 220 may also decrease in the distal direction (FIG. 11.) The dissector tip 220 has a through bore 261 that extends through the tapered distal face 260. The through bore 261 allows a clinician to lasso or grasp the dissector tip 220 with a suture to allow the clinician to more accurately move and position the tool assembly 200 within a body cavity.

FIGS. 15 and 16 illustrate the dissector tip 220 in a first configuration (FIG. 15) and in a second configuration (FIG. 16). In the first configuration (FIG. 15), the dissector tip 220 defines a longitudinal axis that defines an acute angle Ω with the longitudinal axis “X” of the tool assembly 200. As discussed above, it is envisioned that angle Ω can be substantially the same as angle β defined by the tissue guide surface 160 of the cartridge assembly 112. In the second configuration, the dissector tip 220 defines a longitudinal axis that is substantially parallel to the longitudinal axis “X of the tool assembly 200. In order to move the dissector tip 220 between the first and second configurations, a clinician can apply a force to the dissector tip 220 to disengage the protrusion 240 from one of the respective spaced recesses 256a and 256b and pivot the dissector tip 220 about the clevis pin 236 to position the protrusion 240 in the other recess 256a or 256b of the dissector tip 240. For example, when the dissector tip 220 is moved from the first configuration (FIG. 15) to the second configuration (FIG. 16), the clinician applies a force on the dissector tip 220 in the direction indicated by arrows “K” to pivot the dissector tip 220 about the clevis pin 236 in the direction indicated by arrow “L” to remove the protrusion 240 from the recess 256b and move the protrusion into the recess 256a. Although only two configurations are shown in the figures, it is envisioned that the dissector tip 220 may be moved to multiple positions. In that respect, it is envisioned that two or more spaced recesses 256 can be provided to receive the protrusion 240 to retain the dissector tip 220 in multiple different angular positions in relation to the longitudinal axis of the tool assembly 200.

Although the drawings only show retaining structure including a protrusion and a plurality of recesses, it is also envisioned that a variety of different types of retention structures can be provided to releasably retain the dissector tip in different angular positions. Further, the retaining structure can be supported on or integrally formed with either or both of the dissection tip 220 and the anvil assembly 214.

Persons skilled in the art will understand that the devices and methods specifically described herein and illustrated in the accompanying drawings are non-limiting exemplary aspects of the disclosure. It is envisioned that the elements and features illustrated or described in connection with one exemplary embodiment may be combined with the elements and features of another without departing from the scope of the disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. A surgical stapler, comprising:
an elongate body defining a proximal portion and a distal portion; and
a tool assembly defining a longitudinal axis and comprising:
a cartridge assembly;
an anvil assembly pivotably coupled to the cartridge assembly such that the tool assembly is moveable between an open position and a clamped position; and
a dissector tip comprising a tip beam with a proximal portion and a distal portion, with the proximal portion fixedly secured to the anvil assembly, and with the distal portion being deformable relative to the longitudinal axis to define at least two angled configurations of the dissector tip for use during a surgical procedure.

2. The surgical stapler of claim 1, wherein the dissector tip further comprises a tip beam cover at least partially surrounding the tip beam.

3. The surgical stapler of claim 2, wherein the tip beam cover defines a vertical thickness that decreases toward a distal direction with respect to the longitudinal axis.

4. The surgical stapler of claim 3, wherein the tip beam cover further defines a lateral width that decreases toward the distal direction.

5. The surgical stapler of claim 2, wherein the tip beam cover envelops the tip beam and defines an atraumatic configuration with rounded edge surfaces and a distally-tapering width.

6. The surgical stapler of claim 1, wherein the at least two angled configurations comprises a first configuration with the dissector tip substantially aligned with the longitudinal axis, and a second configuration with the dissector tip defining an acute angle with the longitudinal axis.

7. The surgical stapler of claim 6, wherein the deformable distal beam portion is manually bendable to define the first configuration and the second configuration.

8. The surgical stapler of claim 7, wherein the distal beam portion includes a material with a retaining property such that, after bending the distal beam portion to a desired configuration, the material retains the distal beam portion in the desired configuration.

9. The surgical stapler of claim 1, wherein the tip beam comprises a metallic material, and wherein the tip beam cover comprises a biocompatible material with at least one of a polymeric material, metal, ceramic, or an elastomeric material.

10. A tool assembly for a surgical stapler, the tool assembly comprising:
a cartridge assembly;
an anvil assembly pivotably coupled to the cartridge assembly such that the tool assembly is moveable between an open position and a clamped position; and
a dissector tip comprising a tip beam with a proximal portion and a distal portion, with the proximal portion fixedly secured to the anvil assembly, and with the distal portion being deformable, relative to a longitudinal axis of the tool assembly, to define at least two angled configurations of the dissector tip for use during a surgical procedure.

11. The tool assembly of claim 10, wherein the dissector tip further comprises a tip beam cover at least partially surrounding the tip beam.

12. The tool assembly of claim 10, wherein the at least two angled configurations comprises a first configuration with the dissector tip substantially aligned with the longitudinal axis, and a second configuration with the dissector tip defining an acute angle with the longitudinal axis.

13. The tool assembly of claim 12, wherein the distal portion is manually bendable to define the first configuration and the second configuration.

14. The surgical stapler of claim 10, wherein the distal portion of the tip beam includes a material retaining property such that, after bending the distal portion to a desired angled configuration, the material retains the distal portion in the desired angled configuration.

15. A tool assembly for a surgical stapler, the tool assembly comprising:
a cartridge assembly;
an anvil assembly comprising a base portion and an anvil portion, the base portion coupled to the cartridge assembly such that the tool assembly is moveable from an open position to a clamped position, and the anvil portion secured to the base portion and including a plurality of staple deforming pockets; and
a dissector tip having a tapered distal surface and comprising a tip beam with a deformable distal beam portion that is manually bendable to define at least one angled configuration relative to a longitudinal axis extending through the tool assembly.

16. The tool assembly of claim 15, wherein the dissector tip further comprises a tip beam cover at least partially surrounding the tip beam and defining the tapered distal surface.

17. The tool assembly of claim 16, wherein the tip beam comprises a metallic material, and wherein the tip beam cover comprises a biocompatible material with at least one of a polymeric material, metal, ceramic, or an elastomeric material.

18. The tool assembly of claim 15, wherein the tip beam comprises a proximal portion fixedly secured to the anvil assembly.

19. The tool assembly of claim 15, wherein the distal beam portion includes a material retaining property such that, after bending the distal beam portion to a desired angled configuration, the material retains the distal beam portion in the desired angled configuration.

20. The tool assembly of claim 15, wherein the at least one angled configuration comprises a first configuration with the dissector tip aligned with the longitudinal axis, and a second configuration with the dissector tip defining an acute angle with the longitudinal axis.
